(19) United States
(12) Patent Application Publication
(10) Pub. No.: US 2008/0076176 A1
(43) Pub. Date: Mar. 27, 2008

(54) De-differentiation and re-differentiation of somatic cells and production of cells for cell therapies

(75) Inventors: Tanja Dominko, Southbridge, MA (US); Raymond Page, Southbridge, MA (US)

Correspondence Address: ROPES & GRAY LLP;PATENT DOCKETING 39/361, 1211 AVENUE OF THE AMERICAS, NEW YORK, NY, 10036-8704, US

(73) Assignee: Advanced Cell Technology, Inc., Worcester, MA (US)

(21) Appl. No.: 11/842,074

(22) Filed: Aug. 20, 2007

Related U.S. Application Data
(63) Continuation of application No. 11/055,454, filed on Feb. 9, 2005.
Application No. 11/055,454 is a continuation of application No. 10/487,963, now abandoned.
(60) Provisional application No. 60/314,657, filed on Aug. 27, 2001.

(51) Int. Cl. C12N 5/00; C12N 5/08

(52) U.S. Cl. 435/377; 435/325

(57) ABSTRACT

The invention provides a method for effecting the de-differentiation of a somatic cell by culturing the cell in the absence of growth factors, cytokines, or other differentiation-inducing agents, and introducing components of cytoplasm of plutipotent cells into the somatic cell and allowing the cell to de-differentiate. The method can be used with somatic cells of any type, from any species of animal. The pluripotent cells may be oocytes, blastomeres, inner cell mass cells, embryonic stem cells, embryonic germ cells, embryos consisting of one or more cells, embryoid body (embryoid) cells, moruia-derived cells, teratoma (teratocarcinoma) cells, as well as multipotent partially differentiated embryonic stem cells taken from later in the embryonic development process. After being de-differentiated, the cell can be induced to re-differentiate into a different somatic cell type. A method for de-differentiating a somatic cell and inducing it to re-differentiate into a cell of neural lineage is disclosed.

### RELATED APPLICATIONS

This application claims benefit of priority to U.S. Ser. No. 60/314,657 filed on Aug. 27, 2001 which is incorporated by reference in its entirety herein.

### FIELD OF THE INVENTION

The present invention provides novel methods for de-differentiating adult somatic cells into multi-potential stem-like cells without generating embryos or fetuses. Cells developed using the present invention can then be differentiated into neuronal, hematopoietic, muscle, epithelial, and other cell types. These specialized cells have medical applications for treatment of degenerative diseases by “cell therapy”.

### BACKGROUND

Today, the vast majority of degenerative diseases are treated by drugs or symptomatic therapies (e.g., alleviation of pain) due to lack of available patient-compatible cells or tissues that could replace damaged tissue or repair the lesions induced by a given disorder. Current cell-based therapeutic approaches being developed involve either allogeneic cells derived from human embryonic stem cells or xenogeneic cells derived from pigs. Examples of these approaches for Parkinson's disease are differentiated human neurons (Geron) and fetal pig neural cells (Diacrin). Although these strategies hold scientific promise, they suffer from major limitations. First, there is considerable controversy over the use of human embryos for stem cell research and development. Second, the use of pig cells suffers from potentially unknown issues involving the transmission of porcine-borne pathogens to humans. Third, both of these strategies require the use of immuno-suppression, which increases the risk of infections.

There is at present a great need for an efficient method to derive multi-potential stem-like cells from a patient's own somatic cells. For example, 15.7 million people (5.9% of the population) in the United States have diabetes. Each day approximately 2,200 new cases of diabetes are reported, and nearly 800,000 people will be diagnosed this year. Diabetes is the seventh leading cause of death in the United States and is a chronic disease that has no cure. Debilitating medical conditions caused by diabetes include kidney failure, blindness, heart attack, and stroke. It costs an estimated $140 billion per year to treat diabetes-related illnesses in the United States. It is more difficult to predict the indirect costs of the disease, which are those associated with worker productivity and societal contributions. Autologous cell therapy, which would replace lost pancreatic cells in a single medical procedure, could eliminate most of these costs. The present invention offers a means to cure, not just treat, the disease. Furthermore, the ability to de-differentiate somatic cells to a multi-potential state, provides the opportunity to treat many of the secondary illnesses associated with diabetes as well. The advantage of the present invention over other allogeneic cell therapy-based approaches is a further reduction in complications and associated costs of histo-incompatibility. Of course, the most immediate and vital benefit of the cell therapies made possible by present invention, although not quantifiable, is the unprecedented improvement in quality of life for patients suffering from incurable degenerative diseases.

### DESCRIPTION OF THE DRAWINGS

FIG. 1: Proliferating bovine adult skin fibroblasts growing on 100 mm tissue culture dishes at about 90% confluence.

FIG. 2: Colonies formed by bovine adult fibroblasts four days after the cells were electroporated with high speed xenopus oocyte extract; the cell colonies are morphologically similar to embryonic stem cell colonies.

FIG. 3A: Cells derived from bovine adult fibroblasts electroporated with Xenopus oocyte extract—the cells are beginning to display a neuronal phenotype with a “phase bright” appearance of the cell body.

FIG. 3B: Bovine fibroblast-derived cells that are beginning to display a neuronal phenotype.

FIG. 4: Bovine fibroblast-derived cells with a neuronal phenotype and axonal-like processes. The cells were obtained by culturing the cells shown in FIGS. 3A/B for 3 days in DMEM/F12 ITS with 10 μg/ml Nerve Growth Factor.

FIG. 5: Bovine fibroblast-derived cells with a neuronal phenotype and axonal-like processes that appear to be in contact with one another. The cells were obtained by culturing the cells shown in FIGS. 3A/B for 3 days in DMEM/F12 ITS with 10 μg/ml Nerve Growth Factor. (FIG. 5).

FIG. 6: Bovine fetal pancreas primary cell culture 3 days after isolation. Cells either plated down (A) or remained in suspension in aggregates (B). Pancreatic cells four weeks after initiation of culture (C). Bovine fibroblast primary cell cultures (controls, D) were dissociated by trypsinization and electroporated with CytoTracker Blue (Molecular Probes, Eugene, Oreg.) prelabeled bovine oocyte lysate. After the electroporation, cells were plated on gelatin coated cell culture dishes and examined for the presence of CytoTracker Blue 24 hours later (E-phase, F-fluorescence using UV excitation). After 1 week in culture, the cells started forming colonies resembling stem cell aggregates (G), which increased in size during the following 2 weeks (H, I). All images were taken at 100×, recorded with DAGE-MTI camera and printed on a UVP printer. Images were scanned into Adobe Photoshop and pseudo-colored.

The remaining cells were plated in 3 replicate 60 mm dishes of cells. After 3 days, the medium was changed to 1) DMEM/F12 ITS; 2; and 3) Neurobasal Medium A (NBA, Clonetics) with 10 μg/ml NGF. The cells treated with DMEM/F12 ITS alone displayed a phenotype similar to that observed before.

### DESCRIPTION OF THE INVENTION

The present invention exploits the fact that all of the somatic cells of an individual contain the genetic information required to become any type of cell, and when placed into a conducive environment, a terminally differentiated cell's fate can be redirected to pluripotentiality. This fact has been exemplified by the success of somatic cell nuclear transfer experiments in non-human mammals. As normal development proceeds, the gene expression profile of a cell becomes restricted and regions of the genome are stably inactivated such that, under normal conditions, the cell cannot rejuvenate. It is well-established that cell type-specific gene expression can be altered by environmental insults (as in wound healing, bone regeneration, and cancer). The present invention provides cells with intracellular and environmental clues that will induce changes in nuclear function and consequently, change the cell's identity. Using the present invention, cytoplasm from known pluripotent cell types, such as human teratocarcinoma cells, spermatogonia, mature frog, and mammalian oocyte cytoplasm extract is incorporated into somatic cells by electroporation or by BioPorter® (Gene Therapy Systems, San Diego, Calif.). After incorporation, cells are cultured using conditions that support maintenance of de-differentiated cells (i.e. stem cell culture conditions). The dedifferentiated cells can then be expanded and induced to re-differentiate into different type of somatic cells that are needed for cell therapy; for example, into glucose responsive, insulin-producing pancreatic beta cells.

The present invention permits the memory of an adult differentiated somatic cell to be replaced with its long forgotten embryonic memory by manipulating the intra- and extra-cellular environment. By providing an adult somatic cell with factors present in mature oocyte cytoplasm and/or factors present in other known pluripotent cell types (e.g., spermatogonia, teratocarcinoma cells), the invention restores the cells' epigenetic memory to a state similar to that of pluripotent stem cells (without creating an embryo). The invention provides a means for (1) determining the minimal effective quantity of oocyte cytoplasmic lysate/extract required for reprogramming, and (2) preparing high-speed extracts from lysates to eliminate the mitochondrial and nuclear contribution from the “reprogramming matrix” and make it semi-defined. The high-speed extract can be fractionated and individual fractions tested for reprogramming ability, leading to development of a product for reprogramming somatic cells.

In practicing the present invention, no embryos or fetuses of any species are ever created or used and no mixing of human and non-human mitochondrial or genomic DNA ever occurs. All the methods of the invention can be performed in vitro and sources of reprogramming cytoplasm are available from local slaughterhouses (bovine oocytes and spermatocytes), Xenopus oocytes (in house, IACUC approved), or from commercial sources (teratocarcinoma cells from ATCC).

The object of the present invention is to develop technology to change the nuclear function of one type of highly specialized somatic cells, e.g. skin fibroblasts, into that of another type, e.g., fully functional pancreatic islets, via a “novel” pluripotent cell intermediate. The invention does not utilize embryonic or fetal tissues to accomplish the change in function and can be designed for individual patients using their own cells.

The invention exploits the fact that all of the cells of an individual contain the genetic information required to be expressed by any cell type when placed into a conducive environment (as shown by somatic cell nuclear transfer experiments). Most of this information becomes repressed as differentiation proceeds and remains stably inactivated in all differentiated cell types. It is well established that expression of cell type-specific genes is determined by environmental signals and can be altered by environmental insults (as in wound healing, bone regeneration, and cancer). The present invention provides cells with intracellular and environmental clues that will induce change of nuclear function and consequently change cells' identity. In one embodiment of the invention, cytoplasmic extract from known pluripotent cell types, such as human teratocarcinoma cells, spermatogonia, and mature frog and mammalian oocytes, is delivered into somatic cells by electroporation or by BioPorter® (Gene Therapy Systems, San Diego, Calif.). After delivery, the cells are exposed to an environment that supports de-differentiated cell types; e.g., stem cell culture conditions. Upon expansion to numbers sufficient for several differentiation pathways, the cells are directed to re-differentiate; for example, into pancreatic islet cells.

As shown by the success of somatic cell nuclear transfer, the ability to erase the memory of an adult differentiated somatic cell and replace it with it's long forgotten embryonic memory is limited only by the ability to manipulate the intra- and extra-cellular environment. By providing the nucleus of an adult somatic cell with factors present in mature oocyte cytoplasm (without creating an embryo) and/or factors present in other known pluripotent cell types (spermatogonia, teratocarcinoma cells), the present invention alters nuclear memory and induces nuclear changes that are commonly observed in pluripotent stem cells. Benefits and advantages of the invention include the following:
(i) No need for human embryos or fetal tissue. With the present invention, embryos do not have to be used, created, or destroyed to generate pluripotent cells, thus eliminating ethical concerns.
(ii) No need for patient immuno-suppression. In most cases, extended graft survival can only be expected when combined with pharmaceutical immuno-suppression. A preferred method of long-term and lasting treatment using cell-based therapy is to use cells originally derived from the patient.
(iii) No health risks due to possible transmission of animal viruses. Since no component of the animal genome is ever used in the invention, potential threats due to animal genomic DNA sequences are not a concern.
(iv) No mitochondrial incompatibility. Mitochondrial DNA is removed from the reprogramming matrix by ultracentrifugation.
(v) No need for pharmacological therapy. Cell transplantation can be used alone and does not have to be supported by any pharmacological agents.
(vi) Few or no side effects. Autologous cell transplantation is unlikely to induce adverse side effects.
(vii) No tolerance/resistance induction by therapy. Autologous cell transplants are not expected to induce resistance and if required, repeated cell transplantation is feasible.
(viii) Short cell generation time. This invention contrasts with embryonic methods, which have yielded only small numbers of starting stem cells (between 10-15 cells from a blastocyst). Since large numbers of cells can be harvested from individual patients (a single, common source of stem cells is not required any longer) as starting material, the degree of in vitro proliferation is only what is needed to de-differentiate them and generate enough cells for the clinical application.
(ix) Cure, not only treatment. The present invention will significantly reduce the cost of cell therapy by eliminating the need for immuno-suppression of the patient to reduce acute and hyperacute rejection. The need for repeated transplantation procedures will also be alleviated, reducing the indirect cost of disease treatment.
(x) Model. Presumptive human pancreatic beta cells can be tested by transplantation into SCID mice as described (Lanza et al., 1997) and do not require a non-human primate model.
   Abbreviations used in Application
   3-D—three dimensional
   5′UTR—5′-untranslated region
   ACT—Advanced Cell Technology
   Alpha 1AT—alpha 1 anti-trypsin
   ANOVA—analysis of variance
   ATCC—American Type Culture Collection
   bFGF—basic fibroblast growth factor
   bHLH—basic-helix-loop-helix
   CAMs—cell adhesion molecules
   CDk2 cell cycle kinase
   DMEM—Dulbecco modified minimum essential medium
   DMSO—dimethylsulfoxide
   DTZ—dithizone
   EGM—endothelial growth medium
   EIA—adenoviral protein
   EC—extra cellular
   FACS—fluorescence assisted flow cytometry sorting
   FCS—fetal calf serum
   FFA—free fatty acids
   G0/G1—gap phases of the cell cycle
   GCT44—human yolk sack teratoma cell factor
   GFP—green fluorescent protein
   H1—histone H1
   HDL—high-density lipoproteins
   HDM—hormone-defined medium
   HGF—hepatocytes growth factor
   HGM—hepatocytes growth medium
   HPLC—High performance liquid chromatography
   IACUC—Institutional Animal Care and Use Committee
   IAPP—anti-islet amyloid peptide
   ICC—immunocytochemistry
   IVF—in vitro fertilization
   Kb—kilobase
   LDL—low-density lipoproteins
   LIF—leukemia inhibiting factor
   LN₂-liquid nitrogen
   NGF—nerve growth factor
   NuMA—nuclear matrix associated protein
   Oct4GFP—a transgene: Oct4 promoter (transcription factor) driving GFP (Green Fluorescent Protein) expression
   PEG—polyethylene glycol
   PERVS—Porcine endogenous retroviruses
   PL—phospholipids
   RT-PCR—reverse transcription-polymerase chain reaction
   SCID—severe combined immune deficiency
   TRITC—isothiocyanate

This invention essentially provides a method for de-differentiation of one type of somatic cells into pluripotent stem-like cells using a semi-defined cell-free system in vitro. The invention provides a cell-free reprogramming matrix that will reliably direct de-differentiation of adult differentiated human cells into a stem-like cell type. Stem-like cells are then induced to differentiate into desired somatic cell type. This process provides autologous (isogeneic) cell types for cell transplantation in the same individual that donated the initial somatic cell sample. The present invention circumvents problems of histo-incompatibility that exists with competing cell therapy strategies, and shortens significantly the time required for the “new” cells to be available for therapy and does not use embryo or fetus intermediaries as vehicles for reprogramming.

The invention also includes methods for characterization and maintenance of the newly de-differentiated cells, stable cell morphology and analysis of cell-specific gene and protein expression; and induced re-differentiation into cells of another type.

The present invention provides for efficient reprogramming and de-differentiation of somatic cells; maintenance of de-differentiated state in vitro; determining the ability of cells to differentiate upon induction, and the assessment of newly induced differentiated cell types to exhibit proper function upon cell transplantation. Aspects of the invention include characterizing both de-differentiated and newly induced cell types for their gene expression, protein expression, secretory function, presence of cell surface antigens, ability to proliferate, and karyotype stability. Specific aspects of the invention are described in detail below.

Preparing and Characterizing High-Speed Extracts (Reprogramming Matrix)

Components of reprogramming machinery are clearly present in mature, metaphase II arrested mammalian oocytes, as shown by the successes of nuclear transplantation experiments. Various types of adult somatic nuclei from several species have been reprogrammed using an oocyte cytoplasm where the nucleus acquired totipotency, and reconstructed embryos developed into healthy offspring upon transfer into recipient animals (reviewed by Pennisi and Vogel, 2000). An approach to conceptually related to reprogramming after nuclear transfer into oocytes is the study of changes in nuclear function that occur after the fusion of two distinct somatic cell types into a heterokaryon. A gene that is normally active only in a given cell is often inactivated upon fusion of that cell with a different type of cell or with an undifferentiated cell (Kikyo and Wolffe, 2000). Similarly, activation of a new gene can occur by induction of pluripotent cell-specific transcription factors that in turn might activate a diverse group of genes downstream (Hardeman et al., 1986).

Xenopus extracts have been used extensively for examination of mammalian somatic cell gene activity during the past 40 years. After incubation of a nucleus in oocyte extracts, a considerable amount of protein is taken up into the nucleus (Merriam, 1969). This is accompanied by nuclear swelling and a decrease in the amount of heterochromatin in the somatic nucleus. Remarkably, over 75% of pre-existing somatic nuclear protein is lost, probably due to the active oocyte nucleoplasmin. In addition to nucleoplasmin, energy-dependent chromatin remodeling machinery is probably required for reprogramming nuclei (Blank et al., 1992). Such energy-dependent process may involve ATPases, DNA polymerases, or dedicated chromatin-remodeling machines, such as SWI2/SNF2 superfamily. Indeed, it has been shown that nucleosomal ATPase ISWI has an important role during this process (Kikyo et al., 2000). The results of experiments of these and other researchers suggest that cells maintain continuous regulation of a plastic differentiated state in which all of the genes are continually regulated by trans-acting factors that either activate or repress their transcription. (Blau and Baltimore, 1991). The process of transcription requires considerable remodeling of chromosomal structure, such as that which occurs in Xenopus egg cytoplasm (Kikyo and Wolffe, 2000). The present invention demonstrates that reprogramming matrix components can be isolated in a semi-pure protein complex form from oocytes and pluripotent cell types and used to revert nuclear function of somatic cells.

Preparation of High-Speed Metaphase II Xenopus Pocyte Extract.

Mature Xenopus oocytes are obtained from superovulated female frogs and low and low speed and high-speed extracts can be prepared as described (Blow and Laskey, 1986). Oocytes are placed in High Salt Barth solution (110 mM NaCl, 2 mM KCl, 1 mM MgSO₄, 0.5 mM Na₂HPO₄, 2 mM NaHCO₃, 15 mM Tris-HCl, pH 7.4) and processed within 2 hours. The eggs are dejellied in 2% cystein (pH 7.8) and washed several times in 20% modified Barth Solution (20% MBS: 18 mM NaCl, 0.2 mM KCl, 0.5 mM NaHCO₃, 2 mM Hepes-NaOH, pH 7.5; 0.15 mM MgSO₄, 0.05 mM Ca(NO₃)₂, 0.1 mM CaCl₂). The eggs may then be activated for preparation of interphase extract (e.g., by 0.5 μg/ml Ca-ionophore A23187 for 5 min), or used un-activated for the extract preparation. They are washed in ice-cold extraction buffer: 50 mM Hepes-KOH (pH 7.4), 50 mM KCl, 5 mM MgCl₂, 2 mM β-mercaptoethanol, 3 μg/ml leupeptin and 10 μg/ml cytochalasin B. Washed eggs are pooled into cooled centrifuge tubes, the excess buffer is removed and the eggs are crushed by centrifugation in a swinging bucket rotor (e.g., Sorvall® AH-650) at 9,000 rpm at 4° C. for 15 minutes. This produces 4 major fractions: a dense insoluble plug of yolk platelets and pigment, a golden-brown cytoplasmic layer, a lighter translucent cytoplasmic layer, and a yellow plug of lipid. The golden colored cytoplasmic layer is removed with a cooled Pasteur pipette and centrifuged in the same rotor at 9,000 rpm at 4° C. for 15 minutes again in order to remove residual debris. The final protein concentration in the extract ranges around 45 mg/ml. High speed extract is prepared from the golden cytoplasmic layer by centrifugation at 100,000 g for 60 minutes. A translucent pellet of polyribosomes and glycogen is found at the bottom of the tube. Heavy membranes sediment above. The cytoplasmic layer is removed and used to in the procedures to effect de-differentiation. To preserve cellular proteins and their activity, all the procedures are carried out at 4° C.

Extracts are prepared from bovine oocytes, teratocarcinoma cells and spermatogonial cells using similar methods. Every batch of extract is screened for the presence of genomic and mitochondrial DNA by Hoechst 33342 and MitoTracker DNA staining.

Protein content of extracts is determined by established protocols (BioRad®, Hercules, Calif.). The extract is fractionated by HPLC using Superdex® column, which separates proteins based on their size and shape. Each fraction is collected and tested individually for its reprogramming activity.

Collecting and Analyzing Data.

The extracts can be characterized for the presence of molecules that have been shown in intact oocytes to be important during normal fertilization and embryonic development. For example, levels of histone H1 kinase cdc2 (relating to preservation of the metaphase state) and MAP2 kinase and their dynamics and persistence in cell-free extracts prior to hybridization by Western blotting can be determined, as well as quantities and the phosphorylation state of CDK2, cyclin A, Cyclin B, cyclin E, cdc25, p53, nucleoplasmin, histones, RNA and DNA polymerases, Oct4 transcription factor and E1A-like protein, which can be routinely monitored by Western blotting. The molecular profile of each batch of extract can be standardized so that known dilutions of proteins/activity are present in the hybridization matrix. A minimum effective dose is determined as that giving 50% of hybridized cells showing change of nuclear function (down-regulation of donor cell-specific genes) within 48 hours, and by induction of Oct4GFP fluorescence.

Delivery of Extracts into Patient's Somatic Cells.

In order to introduce large molecules into living cells, the plasma membrane needs to be perturbed. There are several published protocols that can achieve this goal with various degrees of efficiency; for example, electric fusion, electroporation, polyethylene glycol treatment (PEG), and liposomes are some of these protocols. In addition, the following two approaches can be used to effect extract delivery:

1. The BioPorter® protein delivery reagent (Gene Therapy Systems, Inc.) is a unique lipid based formulation that allows the delivery of proteins, peptides or other bioactive molecules into a broad range of cell types. It interacts non-covalently with the protein creating a protective vehicle for immediate delivery into cells. It fuses directly with the plasma membrane of the target cell. The extent of introduction can be monitored by TRITC-conjugated antibody uptake during hybridization. This is easily monitored using low light fluorescence on living cells. Molecules that have been successfully introduced into various cell types include high and low molecular weight dextran sulfate, B-galactosidase, caspase 3, caspase 8, granzyme B and fluorescent antibody complexes.

2. Electroporation of plasma membrane, a technique commonly used for introduction of foreign DNA during cell transfections, can also be used. This method introduces large size, temporary openings in the plasma membrane, which allows free diffusion of extracellular components into cells.

The methods of the present invention can be used to effect de-differentiation and re-differentiation of any type of germ cell or somatic cell. Examples of cells that may be used include but are not limited to fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, osteocytes, macrophages, monocytes, and mononuclear cells.

The cells with which the methods of the invention can be used can be of any animal species; e.g., mammals, avians, reptiles, fish, and amphibians. Examples of mammalian cells that can be de-differentiated and re-differentiated by the present invention include but are not limited to human and non-human primate cells, ungulate cells, rodent cells, and lagomorph cells. Primate cells with which the invention may be performed include but are not limited to cells of humans, chimpanzees, baboons, cynomolgus monkeys, and any other New or Old World monkeys. Ungulate cells with which the invention may be performed include but are not limited to cells of bovines, porcines, ovines, caprines, equines, buffalo and bison. Rodent cells with which the invention may be performed include but are not limited to mouse, rat, guinea pig, hamster and gerbil cells. Rabbit cells are an example of cells of a lagomorph species with which the invention may be performed.

Specific somatic cells with which the invention can be performed are human skin fibroblasts transgenic for mouse Oct4 promoter-driven GFP gene. The mouse Oct4 promoter can drive GFP expression in porcine and bovine preimplantation embryos (Kirchhof, et al., 2000). Oct4 is the only known molecular marker of pluripotency that has been shown to be absolutely required for normal development of pluripotent mammalian inner cell mass during early embryogenesis. Pluripotent embryos and embryonic stem cells as well as embryonic-derived tumors are the only tissues in mammals that show expression of this gene (Schöler et al., 1991, Pesce and Schöler, 2000). For example, the mouse Oct4 promoter and its regulatory 5′UTR (8 Kb-H. Schöler) can be used to direct expression of GFP gene as a marker of successfully de-differentiated cells.

Introducing Extract Using Bioporter® Reagent

Donor somatic cells can be grown as monolayers in tissue culture dishes and synchronized in G1 phase of the cell cycle by methods described in literature (Leno et al., 1992). For example, growing primary cultures can be synchronized by an initial S phase block for 20 hours with 2.5 mM thymidine, followed after a 5 hour interval by a 9 hour mitotic block by demecolcine. Three hours after release from demecolcine, the cells synchronously enter G1 phase. BioPorter® reagent coated cell extract can be added to the cultured cells and incubated 4 hours at 37° C. The cells that incorporated extract can be identified and separated from the other cells, e.g., by washing and sorting them using fluorescence assisted flow cytometry (FACS) with detection of the presence of the TRITC-labeled control immunoglobulin in cells. Positive, fluorescent cells can be are collected, the medium replaced with stem cell medium, and the cells cultured using conditions designed for stem cells.

Introducing Extract Using Electroporation

Alternatively, the extracts can be electroporated into the target cells; e.g., using methods developed for hybridoma formation. The electroporation procedure introduces holes in the plasma membrane that permit entry of large protein extracellular molecules into cells without the requirement for an active uptake. Electroporation parameters are tested and optimized for the specific donor cell type.

As stated above, the extent of delivery can be monitored by the presence of TRITC-conjugated antibody inside the donor cells after the 4-hour hybridization period. Optimal parameters, e.g., concentrations of BioPorter®, the cell extract, and duration of treatments, can be determined experimentally in order to achieve 50% uptake. Uptake can be monitored by live time-lapse video imaging on an inverted microscope, equipped with an environmental chamber. TRITC-positive cells can be separated from non-positive cells by flow cytometry and used for putative stem cell culture. The expression of Oct4-GFP in live cells can be measured to evaluate the timing and progress of the de-differentiation process occurring within the treated cells.

The proportions of cells that take up extract may exceed 50% using either electroporation or BioPorter® system. Different donor cell types may require unique electroporation and/or BioPorter® conditions; these can be determined experimentally. The procedure can introduce amounts of reprogramming matrix sufficient to effect de-differentiation into the majority of manipulated cells; consequently high numbers of putative stem cells can be obtained in each experiment. The introduced reprogramming matrix is retained by the cells regardless of the method by which it is introduced. Activity of the reprogramming matrix lasts at least 48 hours after hybridization. During this time cells can be kept in a maintenance medium that prevents growth and DNA replication in order to extend the duration of G1 reprogramming phase. Cells synchronized in G1 will be most likely affected by the matrix and the most likely to revert into stem cells (Campbell et al., 1996). After reprogramming, the cells re-enter the cell cycle, retain TRITC fluorescence (indicative of non-leakage) and continue cycling in a manner representative of stem cells. At the time of de-differentiation GFP positive (green) cells are observed, and FACS will separate the GFP positive cells from the rest.

The efficiency of delivery using the BioPorter® system depends on the cells' density and/or confluence, delivery time, amount of protein in the extract to be delivered, concentration of the protein solution during preparation of the complexes (BioPorter®-protein complexes) and the hydration volume for BioPorter® reagent. Accordingly, these parameters are can adjusted and the protocol optimized for delivery into 50% or more of the target cells. If protein concentration of the cytoplasmic extract is determined to be too low, the extracts can be lyophilized and the concentration of proteins optimized by dry weight. The fraction of the lysate or a combination of 2 or more fractions that is/are responsible for the reprogramming can be identified by HPLC fractionation of the extract and testing of the fractions individually for their reprogramming ability. The invention includes identifying and using those fraction(s) of the whole extract that are required to effect active reprogramming (de-differentiation).

Different donor cell types are likely to require different amounts of active extract and/or different duration of delivery in order to de-differentiate. Accordingly, different somatic cell types can be examined for their susceptibility for reprogramming, e.g. skin fibroblasts, keratinocytes, hair follicle cells, white blood cells and muscle cells. Upon demonstration that a certain cell type is particularly amenable to reprogramming, that cell type can then be used in subsequent experiments. Cell extracts obtained from oocytes, teratocarcinoma cells and spermatogonia are expected to display different reprogramming capacity. Their reprogramming capacity will be correlated with the ease of preparation, ability to generate sufficient volumes and protein quantity, repeatability of preparation, consistency of reprogramming activity and ease of delivery. Optimizing these factors is within the level of skill in the art.

In addition to BioPorter® and electroporation, reporgramming extracts can be introduced into cells using membrane enclosed cytoplasmic fragments from the pluripotent cell types mentioned above; by hybridizing them with donor cells by electrofusion or PEG-mediated fusion.

Evaluating De-Differentiated Cells

Embryonic stem cells retain their pluripotency in vitro when maintained on inactivated fetal fibroblasts in culture. More recently, it has been reported that human embryonic stem cells can successfully be propagated on Matrigel in a medium conditioned by mouse fetal fibroblasts (Xu et al., 2001). Human stem cells can be grown in culture for extended period of time (reviewed by Thomson and Marshall, 1998) and remain undifferentiated under specific culture conditions. De-differentiated cells are expected to display many of the same requirements as pluripotent stem cells and can be cultured under conditions used for embryonic stem cells.

Methods for Evaluating De-Differentiated Cells Include:

1. Monitoring changes in the cells' phenotype and characterizing their gene and protein expression. Live time-lapse video imaging can be used to monitor the uptake of the extracts, changes in cell morphology upon hybridization (or lack thereof), and dynamics of changes induced as well as GFP transgene fluorescence.

2. Screening results can be compared to results obtained with undifferentiated, pluripotent control cells such as monkey parthenogenetic stem cells (Advanced cell technology), or human embryonic stem cells (Wisconsin Alumni Research Foundation, Madison, Wis. and Geron, Inc). Stem cell markers and morphometric and growth characteristics of parthenogenetic cynomolgous monkey embryonic stem cells (Cibelli et al., Nature, in press) match with those published by Thomson et al. (1998) for human embryonic stem cells obtained from in vitro fertilized human blastocyst.

The expression of the following genes of de-differentiated cells and human embryonic stem-like cells can be compared: alkaline phosphatase, Oct4, SSEA-3, SSEA-4, TR-1-60 and TR-1-81 (Thomson et al., 1995, 1998). Assays designed to detect expression of genes specific to the given cell type can be used to confirm the presence of expression in the cells prior to hybridization, and to confirm the absence of expression after hybridization. Self-renewing capacity, marked by induction of telomerase activity, is another characteristic of stem cells that can be monitored in de-differentiating cells (Morrison et al., 1996).

Maintenance of the Undifferentiated State

Mouse fetal fibroblasts can be mitotically inactivated by irradiation and prepared at 5×10⁴ cells/cm² on tissue culture plastic previously treated by overnight incubation with 0.1% gelatin (Robertson, 1987). Fibroblasts can be prepared a day before hybridization construction and cultured in DMEM, supplemented with 20% fetal bovine serum, 0.1 mM mercaptoethanol and 0.1 mM non-essential amino acids and human recombinant LIF.

As an additional means to maintain an undifferentiated state, hybrid cells growing on fibroblast feeder layers, can be supplemented with GCT44 factor (human yolk sac teratoma cell factor; Roach et al., 1993). Gene expression can be determined by RT-PCR, and translation products by immunocytochemistry and Western blotting. Markers for the expression of specific genes in the donor cells can be identified depending on the cell type. For example, the fibroblast surface protein gene can be used as a marker for expression in fibroblasts, etc. RT-PCR assays can be used to demonstrate expression in donor cells and absence of the product is an indication that expression of that gene has been lost. To evaluate de-differentiation, induction of expression of SSEA-3, SSEA-4, TR-1-60, TRA-1-81, alkaline phosphatase and Oct4 can be monitored. Immunocytochemistry can be used to detect gene products. RT-PCR primers and hybridization probes and antibodies for immunocytochemistry and Western blotting are commercially available. Expression of Oct4GFP transgene can be monitored by live fluorescence microscopy.

Telomerase activity is assayed as described by Thompson et al. (1998). The TRAPEZE telomerase detection kit is used (Oncor, Gaithersburg, Md.). About 2000 cells are analyzed at every experimental time point and 800 cell equivalents are loaded in each well of a 12.5% nondenaturing polyacrylamide gel. Reactions are done in duplicates. Finally, cells can be injected into SCID mice and monitored for development of teratomas. After 6 weeks, teratomas are analyzed by histological sectioning and presence of various tissues determined. Assay can also be performed to determine the potential of the cells to induce formation of embryoid bodies and to undergo spontaneous differentiation in culture.

Temporal expression of key marker genes can be monitored at each passage to determine the timing of reprogramming in the hybridized cells. This yields information as to how long it takes for the somatic cell (differentiated state) to de-differentiate with respect to its gene expression profile. Morphology of de-differentiated cells, timing and progression of cell cycles and doubling times can be monitored daily by live time-lapse video imaging in parallel with incubated cultures. In addition, mitotic cells can be shaken off the monolayers and used for gene expression analysis and ICC after different numbers of passages. Their gene expression profile is compared with that of the somatic donor cell type. The length of time de-differentiated cells can be maintained in culture is monitored and any change in morphology or gene expression determined. Observation that the hybridized cells display loss of tissue specific protein and gene markers, display change in morphology and acquire stem cell markers is evidence that the cells have undergone de-differentiation and are suitable for induced differentiation.

De-differentiated cells may be slow cycling, with the majority of the cells in G1 phase of the cell cycle, they may display higher nucleo-cytoplasmic ratio than donor somatic cells, possess poor rhodamine uptake into mitochondria, display telomerase activity that is higher than that in untreated cells; and they will express Oct4-GFP. Different donor cell types may demonstrate a variable ability to revert their nuclear function. Growth requirements are generally similar to those of parthenogenetic stem cells, and so is protein and gene expression. Different extracts may induce various degrees of reprogramming. Oocyte extracts are more likely to induce a change into embryonic-like stem cells, while teratocarcinoma and spermatogonial extracts may be more limiting in their ability to reprogram the cells completely.

Partial if not complete reprogramming can occur within the first 24-48 hours after matrix delivery. The extent of reprogramming depends on the donor cell type, cell cycle stage of donor cells, and extract quality/fraction. Tissues originating from different germ layers may have different ability to undergo reprogramming. Expression of pluripotent markers is expected to continue as long as the hybridized cells are cultured under conditions that will maintain their undifferentiated state. Similarly, telomerase activity is expected to be detectable in de-differentiated cells, evidence that the cells have acquired self-renewing capacity.

A Differentiation Protocol for Pancreatic Islets

Pancreatic cells have been reportedly detected at a low frequency in mixed cell populations derived from induced differentiation of embryonic stem cells (Kahan et al., 2001, Schuldiner et al., 2001). The present invention provides a new approach for inducing and directing pancreatic differentiation.

Directed differentiation of stem cells into endoderm-derived cell lineages has not been describe. Except for the demonstration that NGF and HGF (Schuldiner et al., 2000) induce transcription of some endodermal markers (such as albumin, alpha-feto protein, amylase and alpha 1AT) in addition to markers for ecto- and mesodermal development, there is no published literature on directed endoderm differentiation. Lumelsky et al. (2001) reported in Science that they successfully achieved differentiation of mouse embryonic stem cells into endocrine pancreatic, insulin-secreting cells in vitro by first growing mouse embryonic stem cells into embryonic bodies. This is the first time that a significant proportion of stem cells have been reported to actually follow insulin positive differentiation (35% of all stem cells).

Lateral mesoderm (hematopoietic cells) can transdifferentiate into endoderm (liver cells; Theise et al., 2000); accordingly, pancreatic development is expected to occur in a two-step process.

Cell differentiation is defined and supported by the cell's environment; therefore, it is possible to design extracellular matrix, media and supplement combinations that induce pancreatic development. Bovine fetal pancreatic primary cultures (both monolayers and suspension cultures) as “feeders” for stem cell differentiation and pancreatic extracts as supplements to differentiation medium can be used as substrates/helpers for induced differentiation.

Long-term survival and stability of physiological responses has been afforded only by extracts enriched in extracellular matrix. Matrigel (Brill et al., 1994; Grant et al., 1992) has induced cells into far more complicated physiological states than any known purified matrix component by itself. A major function of the matrix is to allow for assembly of cells into a three-dimensional structure, which is essential for achieving fully normal phenotype and for normal transcription rates of tissue-specific genes (Rodriguez-Boulan and Zorzolo, 1993). Extracellular lateral and basal matrix components can be combined to achieve the most physiological conditions for pancreatic development. Cell adhesion molecules (CAMs), proteoglycans (lateral matrix between the same type of cells), laminin and type IV collagen can be provided as components of basal matrix. Extracellular (EC) matrix can be used in combination with a nutrient rich medium, supplemented with fetal bovine pancreas extract and/or supplemented with bovine fetal pancreatic cells embedded in porous gelatin matrix sandwich. Optimal concentrations of HDL/LDL-high and low density lipoproteins, PL-phospholipids, FFA-free fatty acids, bFGF, heparin proteoglycans and glucocorticoids can be determined by routine assays. Pancreatic extracts are prepared using similar methods as for reprogramming matrix extracts.

Flow cytometric sorting strategies can be developed based on the developing and mature surface antigenic profiles of pancreatic cells. Cells are separated using stem cell surface antibodies to eliminate non-committed cells. Serum-free hormone defined medium (HDM) is used instead of animal serum for all culture in order to allow for reproducibility.

Developing cultures are grown on an inverted microscope in an environmentally controlled chamber and a parallel control in a low oxygen incubator. At regular intervals, images are recorded using live, time-lapse video imaging system (in house) and processed to determine change in morphology and population doubling time.

Imaging data obtained is analyzed by Metamorph (Universal Imaging, PA) and real-time developmental sequence reconstructed for analysis. Cells can be sampled every 24-48 hours for immuno-cytochemistry. They can be spun onto glass slides using Cytospin centrifuge (in house) and assayed for loss of stem cell markers as well as acquisition of endodermal and pancreatic markers, such as insulin I and II, glucagon, PDX-1 transcription factor, somatostatin, alpha-amylase, anti-islet amyloid polypeptide-IAPP, glucose transporter 2, and carboxypeptidase A (Chemicon, Temecula, Calif. and BabCo, Richmond, Calif.). The same samples can be analyzed the presence of specific mRNAs by RT-PCR, and for determination of telomerase activity. Presence of insulin in the cells is detected by dithizone (DTZ) staining (Ricordi et al., 1994). Briefly, 10 mg of DZT is dissolved in 1 ml of DMSO (10 mg/ml stock) and 0.5 mg/ml final solution for labeling made in tissue culture medium, supplemented with 2% FCS. Cells are labeled and red staining indicates presence of insulin. Insulin positive cell are counted followed by determination of the percentage of insulin-positive cells in the total cell population.

Various cell types are generated using the above protocol, however, induction of endoderm-derived cell types is significantly enriched when compared to default differentiation from embryonic bodies (Lumelsky et al., 2001). All three types: endocrine, exocrine and ductal cell types can develop, as a complex, three-dimensional substrate will be provided.

Genes that have been implicated in early determination of pancreatic endocrine lineages include basic-Helix-Loop-Helix (bHLH) transcription factors (IsI1, Nkx2.2, NeuroD/B2, Pax4 and Pax 6; Sander and German, 1997; Edlund, 1998, 1999; St-Ogne et al., 1999) and the PDX1 homeobox gene. If necessary, constructs can be designed with promoters of these genes driving a GFP reporter, and a neomycin trap. Transgenic cells would allow for not only monitoring of cells for expected gene expression but also allow for selection of transgenic cells actively transcribing pancreatic genes to be selected for by neomycin supplemented medium. It is interesting that the same genes are expressed during early neuronal development, which suggests that early development of several tissues may be under similar control. The initiation of pancreatic development and cell-type specification are two of the three levels of development that can be accomplished. The third one (progression of pancreatic development) determines organogenesis and is not anticipated. Initiation is monitored by detection of a beta-cell-specific Hb9 homeobox gene and IsI1/PDX1 gene expression (Odorico et al., 2001). For specification of cell fate, ngn3 gene expression is monitored.

Maintaining Stable Morphology and Function of Newly Differentiated Cells

It is anticipated that cultures of pancreatic cell can be used for transplantation immediately or cryopreserved for later use. It is important to examine cell functionality and lifespan in vitro prior to initiating transplantation studies in mice. Cultures of primary pancreatic cells have been described and we have been successful in culturing fetal bovine pancreatic cells for over 2 months. Cells retain their morphology, remain non-adherent, display classic endocrine morphology with large cytoplasmic vesicles and form colonies indicative of pancreatic islets. They can be subcultured and are well supported without extracellular matrix when grown in hepatocytes (HGM) and endothelial growth media (EGM; both are serum-free; Dominko et al., unpublished). Newly developed pancreatic cells are cultured using the same conditions.

Pancreatic cells are grown at low density in suspension using EGF and HGF media. The cells are sampled at regular intervals and assayed for maintenance of insulin synthesis. At every third to fourth passage, the cells are examined by ICC for continued presence of pancreatic markers, for karyotype stability and telomerase activity. Islets are evaluated by criteria proposed by Ricordi et al. (1994).

Using the de-differentiation methods of the present invention, pancreatic cells can be generated from non-transfected, de-differentiated cells to avoid introducing transgenes into a potentially therapeutic cell population. Alternatively, transgenic donor cells may be used; e.g., to trace the cells during animal testing.

Endocrine pancreatic cells are expected to retain their morphology and function for at least 2 months in culture. Due to their relatively slow growth, we expect telomerase to remain active for extended periods of time and karyotype should remain stable at 2n. However, to alleviate any potential difficulties, pancreatic islets are transplanted into diabetic mice as soon as sufficient cell numbers are available.

Testing Functionality of Pancreatic Islets by Transplantation

For human islets, attempts have been made to ascertain islet viability in vivo by transplantation into nude (SCID) rodents, to avoid rejection. These animals have a deficient immune system due to congenital thymic aplasia and are unable to reject transplanted xenogenic tissue. The first report of transplantation dates to 1974 (Povlsen et al.). Several portions of human fetal pancreas were transplanted subcutaneously and histological examination of the excised tissue two months after transplantation revealed a relatively normal lobular appearance with no sign of rejection. Subsequently, a number of groups reported further success with transplantation of human fetal pancreatic tissue and isolated islets into SCID mice (Ricordi et al., 1988, 1991), made diabetic with streptozotocin. Long-term graft survival and functionality were demonstrated. Upon surgical graft removal, mice returned to a diabetic state

Animal experimental protocol has been submitted to the Institutional Animal Care and Use Committee (IACUC) and we expect the protocol to be approved by July 2001. Experimental diabetes will be induced in 10-12 week old male 12/sv mice by a single intraperitoneal injection of streptozotocin (120-150 mg/kg of body weight) in citrate phosphate buffer; pH 4.5; Sigma Chemical Co. St. Louis, Mo.) (Soria et al., 2000). Stable hyperglycemia (300-600 mg/100 ml) is expected to develop within 48-72 hours. Blood glucose levels will be determined busing a blood glucose analyzer (Glucometer Elite XL, Bayer Corp., Elkhart, Ind.). The animals will be grafted with cells or with a buffer vehicle 24-48 hours after the establishment of stable hyperglycemia. 1-2×10⁶ cells in suspension will be injected per animal under the kidney capsule.

Data Collection and Analysis:

Glucose levels can be monitored every 24 hours after grafting. Each transplanted animal serves as its own control, since it is possible to perform nephrectomy of the kidney bearing the graft and produce a rapid return to the diabetic state. In addition, histological studies of the renal subcapsular grafts provide information on the morphologic integrity and cellular composition of the transplanted islets at the end of the study (52 weeks). Data can be analyzed by 2-way ANOVA (accounting for cell line effect and animal effect) and difference in glucose levels evaluated at P=0.05.

Return to normal glucose levels is expected to occur between two and three weeks after transplantation if the islets retain their functionality (Buschard et al., 1976). Graft function is expected to persist for at least a year (Tuch et al., 1984).

### EXAMPLES

### Example 1

### Preparation of High-Speed Metaphase II Xenopus Oocyte Extract

Mature Xenopus laevis females were superovulated with PMSG and 72 hours later induced to ovulate with hCG. Eggs were collected in cold MMR buffer (100 mM NaCl, 2 mM KCl, 1 mM MgCl2, 2 mM CaCl2, 5 mM Hepes, see Julian Blow, 1993) and washed 2 times with High Salt Barth Solution (NaCl 110 mM, Tris-HCl 15 mM, KCl 2 mM, NaHCO3 2 mM, MgSO4 1 mM, Na2HPO4 0.5 mM), EGTA 2 mM). The jelly coats were removed with cold 2% L-cystein free base (Sigma) with 2 mM EGTA at pH 7.8 (adjusted with 6N NaOH). Eggs were washed in unactivating extraction buffer (KCl 50 mM, Hepes 50 mM, MgCl2 5 mM, EGTA 5 mM, Beta-mercaptoethanol 2 mM), and were packaged into 4.4 ml Sorvall® tubes. Excess buffer was removed, and the eggs were crushed by centrifugation in a swinging bucket rotor at 10,000 rpms for 15 minutes. The cloudy, gray middle cytoplasmic layer was removed and centrifuged at 20,000 rpm for 15 min at 4° C. The translucent layer was removed and diluted 1:6 with extract dilution buffer at 4° C. (KCl 50 mM, Hepes 50 mM, MgCl2 0.4 mM, EGTA 0.4 mM; supplemented just before use with DTT 2 mM, 10 ug/ml aprotinin, leupeptin and cytochalasin B each). The extract was diluted 1:6 with the extract dilution buffer. The extracts were centrifuged again at 30,000 rpm for 1.5 hours at 4° C. Two layers were removed: a translucent layer and a golden layer. These were aliquoted at 50 μl/vial, snap frozen in LN2 and stored at −80 C.

### Example 2

### Preparation of Metaphase II Stage Bovine Oocyte Extract

Mature bovine oocytes were aspirated from freshly collected ovaries and were matured in vitro. The oocytes were collected at 20 hours post maturation and stripped free of surrounding cumulus cells by vortexing in 2.5 mg/ml hyaluronidase (Calbiochem) dissolved in DPBS (Biowittaker). Zonae were removed by incubation in 0.5% w/v pronase (Calbiochem) dissolved in DPBS (Biowittaker) and zona-free oocytes washed through several washes of manipulation medium (Modified ACM, designated ACM-P). The oocytes were resuspended in a small amount of fusion medium (200 oocytes in 20 μl of 0.28 M mannitol, 50 μM MgCl₂, 0.1 mg/ml PVP 40 kD, all Calbiochem) and vortexed at high speed for 3 minutes. The vortexed material was examined under a stereomicroscope to confirm the absence of membrane-enclosed cytoplasmic fragments. Oocyte lysate was prepared freshly for each use and kept on ice until use.

### Example 3

### Preparation of Bovine Adult Skin Fibroblasts

Tissue samples from 2 mm circular ear punch biopsies were received in transport media made of DPBS (Biowhittaker) supplemented with Ciproflaxin® (Mediatech, Cat#61-277-RF). A tissue sample was removed from the container using sterile technique, and placed into a 60 mm falcon petri dish with IMDM (Gibco) and zonkers, fungizone, and pen/strep and allowed to soak for 10 minutes. Using a dissecting microscope, the excess connective tissue was removed and the remaining skin moved to another 60 mm petri dish with above medium. The sample was then placed into a 60 mm petri with about 2 ml of medium and minced into small pieces. Fresh medium was added to the dish to loosen the pieces, then all contents added to a T25 tissue culture flask and the final volume of medium was brought to 3 ml. The sample was incubated at 38.50° C. in 5% CO₂ in humidified air for 10 days without changing medium or moving the flask. The cells were then passaged, first to a T75 flask using Trypsin-EDTA (Gibco), then to 4 T75 flasks, and then were frozen in complete medium with 10% DMSO. Prior to use, cells were thawed at 37 C and centrifuged at 800×g for 4 minutes to remove the cryoprotectant and seeded into a 100 mm culture dish 24-48 hours prior to use. Prior to electroporation, cells were trypsinized and washed in culture medium by centrifugation and suspended in culture medium without serum.

### Example 4

### Electroporation of Xenopus Oocyte Extract into Adult Bovine Skin Fibroblasts

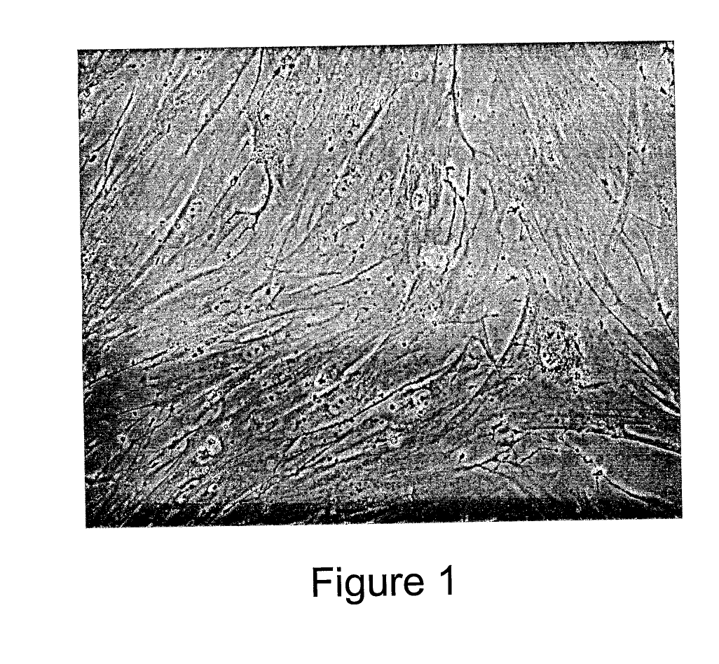

Proliferating bovine adult skin fibroblasts growing on 100 mm tissue culture dishes at about 90% confluence (FIG. 1) were harvested using a 1:1 dilution of trypsin-EDTA (Gibco, Cat# 15400-096) in DPBS without calcium and magnesium. The cells were pelletted by centrifugation and resuspended in fusion medium at 1.0×10⁶ per ml. Twenty μl of cell suspension was added to μl of oocyte lysate and mixed. The cell-lysate mixture was transferred to a 0.5 mm gap width platinum wire electofusion chamber (BTX Model # 450-1) and electroporation was achieved using 2 consecutive DC pulses of 2.0 kV/cm for 15 μsec each. Control experiments were conducted where the oocyte lysate was loaded with 10 μM Cytotracker Blue (Molecular Probes) membrane impermeable cell tracking dye for 45 minutes and washed for 30 minutes. Observation of surviving cells 2 hours after electroporation using fluorescence microscopy confirmed the presence of tracking dye inside the cells, indicating successful transfer of extracellular material into the cells during the electroporation process. Following electroporation, cells were transferred to 1 ml of holding medium (ACM-P) and incubated for 30 minutes at 37 C. Cells were concentrated by centrifugation at 800×g for 4 minutes and transferred to 50 μl drops of KSOM (Cell and Molecular Technologies) in 35 mm petri dishes (Falcon) covered with mineral oil (JT Baker). Cultivation and characterization of bovine adult fibroblasts electroporated with high speed xenopus oocyte extract was as follows:

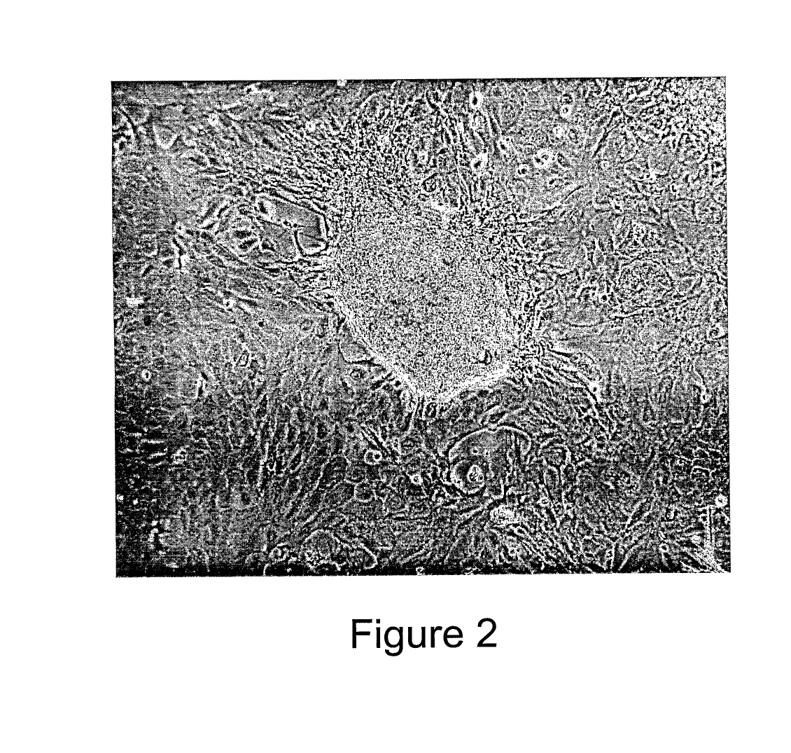

Within 4 days after electroporation, the cells formed colonies morphologically similar to embryonic stem cell colonies (FIG. 2). Cells surrounding the ES-like colonies had an epithelial cell morphological appearance that was different than that of the fibroblasts used as starting material. Attempt to pass these colonies using standard trypsinization procedures failed, which suggests that biochemical changes to the cell's secretion of extracellular matrix had changed as well. Therefore, the colonies were cut into small clumps of cells using a 27 gauge hypodermic needle (Becton Dickinson). Clumps of cells were plated either onto γ-irradiated E14 mouse embryonic fibroblast feeder cells or onto tissue culture plastic without feeder cells. The culture medium was ES cell medium (DMEM, etc., 15% heat inactivated fetal bovine serum, 1% non-essential amino acids, 0.1 mM β-mercaptoethanol, 100 units/ml PennStrep).

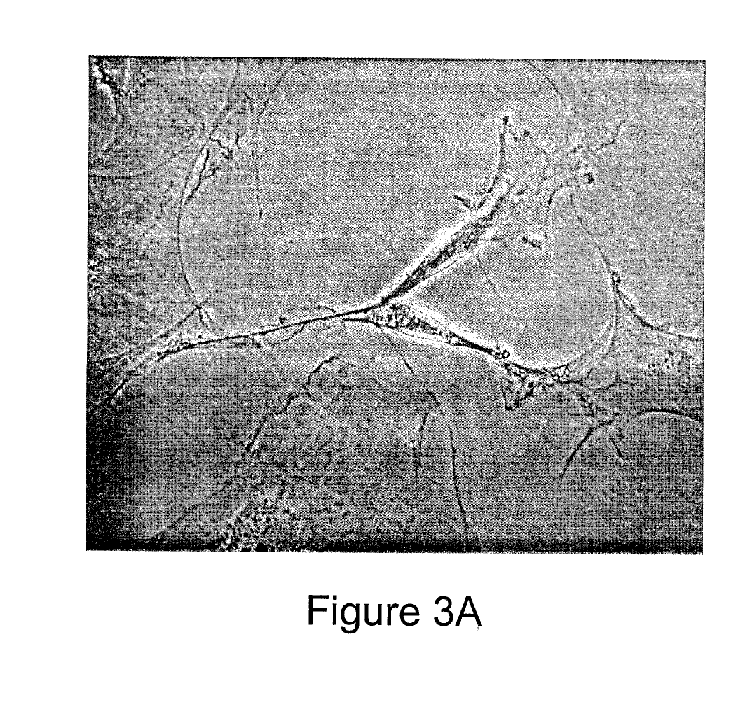

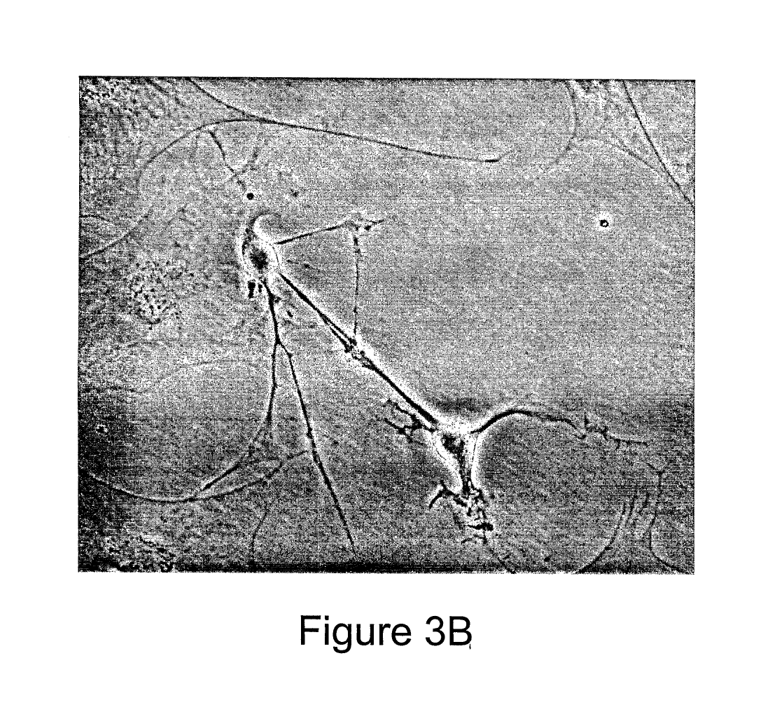

After 7 days, the cells plated on feeder cells failed to proliferate further and were lost upon subsequent subculture, likely due to a poor quality preparation of feeder cells. The cells plated on tissue culture plastic were subcultured into a 100 mm tissue culture dish using a serum-free medium consisting of a 1:1 mixture of DMEM (Gibco) and Ham's F12 nutrient mixture supplemented with Insulin, Transferrin and Selenium (ITS, Gibco). The cells expanded to about 70% confluence and acquired a flattened phenotype and ceased proliferation in this medium. The medium was changed 2× weekly and the cells maintained for 4 weeks. Some of the cells began to display a neuronal phenotype with a “phase bright” appearance of the cell body (FIGS. 3, A & B). The cells were trypsinized and some were re-plated in 24 well plates at about 70% confluence. Three days later, the cells were fixed with 4% parafomaldehyde in DPBS for analysis of cell type-specific markers by immunocytochemistry.

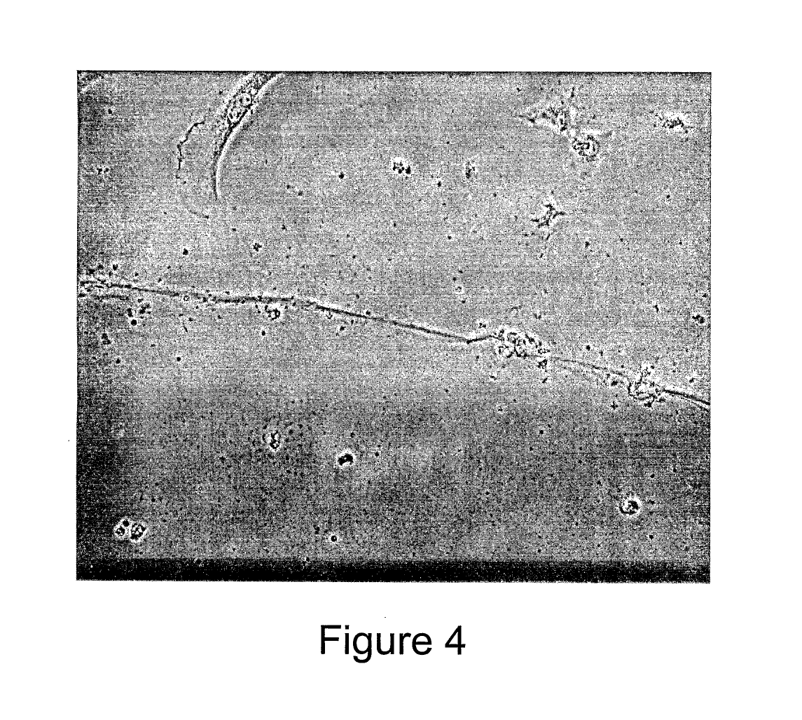

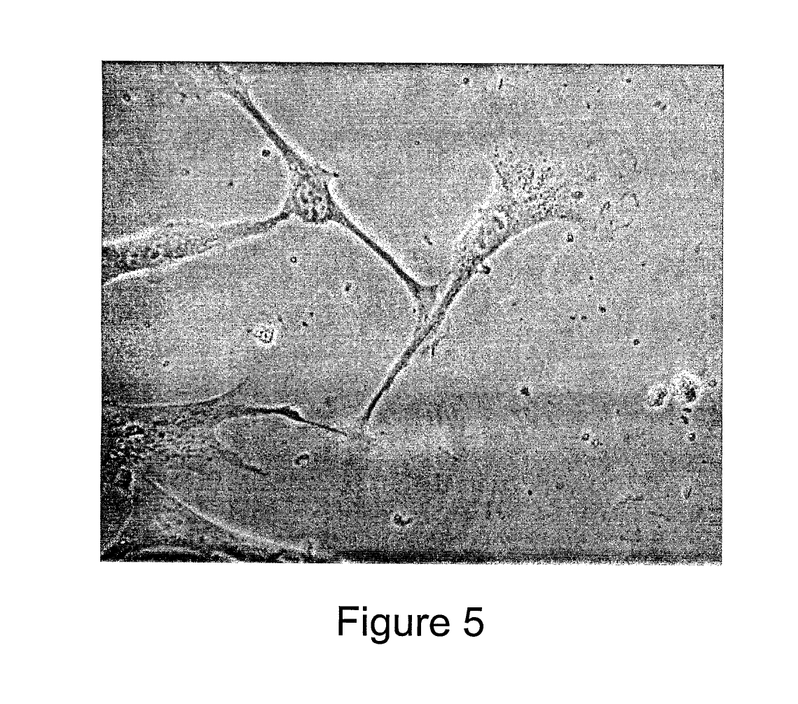

The remaining cells were plated in 3 replicate 60 mm dishes of cells. After 3 days, the medium was changed to 1) DMEM/F12 ITS; 2) DMEM/F12 ITS with 10 μg/ml Nerve Growth Factor (NGF, Supplier XXX); and 3) Nerobasal Medium A (NBA, Clonetics) with 10 μg/ml NGF. The cells treated with DMEM/F12 ITS alone displayed a phenotype similar to that observed before. Cells in DMEM/F12 ITS with NGF had a larger number of cells with a neuronal phenotype as well as an increase in cells with longer axonal-like processes (FIG. 4). In some cases, the processes from adjacent cells appeared to be in contact with one another (FIG. 5). Cells treated with NBA with NGF failed to develop a neuronal phenotype.

### Example 5

### Electroporation of Bovine Oocyte Extract into Bovine Fetal Fibroblasts

The lysate was incubated with 1×10⁶ growing bovine fetal fibroblasts that have been suspended in 40 μl of fusion medium. After mixing, the suspension of cells/lysate was electroporated for 1 msec at 2.0 Kvolts, and the electroporated mixture was placed onto mouse inactivated fetal fibroblasts in embryonic stem cell medium. After culture at 37° C., 5% CO₂ in air for 7 days, the cells formed distinct colonies with appearance similar to those of mouse embryonic stem cells. While we have not yet confirmed the presence of any stem cell markers in these cells, their morphology, characteristic colony growth and nuclear-to-cytoplasmic ratio are indicative of putative stem cells.

### Example 6

Primary pancreatic cell cultures were established from two pancreata obtained from a day 60 and a day 90 bovine fetus. The tissue was removed under sterile conditions, minced with fine scissors and plated in DMEM (Sigma Chemical Co., St Louis, Mo.), supplemented with 10% heat-inactivated fetal calf serum (Hyclone). Primary explants were grown for 3 days in 5% CO₂. Cells were split into two different subcultures. Non-attached cells that maintained a colony appearance and were growing in suspension were passaged into new HGM medium and remained in suspension. The cells that attached during the first three days were trypsinized and subcultured into fresh HGM. These two cell populations remained distinctly different during progressive culture. Non-attached cells continued to proliferate slowly, remained in floating aggregates resembling islets and were viable after over 2 months of culture. Adherent cells displayed different morphology. They clearly formed small clusters, but these clusters were attached to the bottom of the dish and were surrounded by stromal-like fibroblast cells. This demonstrates our ability to maintain pancreatic cultures in vitro.

Our preliminary data demonstrated that introduction of oocyte cytoplasmic lysate into fibroblasts by electroporation induces a change in morphology. Mature bovine oocytes were collected at 20 hours post maturation and stripped free of surrounding cumulus cells by vortexing in 2.5 mg/ml hyaluronidase. Zonae were removed by incubation in 0.5% pronase and zona-free oocytes washed through several washes of medium. The oocytes were resuspended in a small amount of fusion medium (200 oocytes in 20 μl of 0.3 M sorbitol, 50 μM MgCl₂) and vortexed at high speed for 3 minutes. The vortexed material was examined under a steremicroscope to confirm the absence of membrane-enclosed cytoplasmic fragments. The lysate was incubated with 1×10⁶ growing bovine fetal fibroblasts that have been suspended in 40 μl of fusion medium. After mixing, the suspension of cells/lysate was electroporated for 1 msec at 2.0 Kvolts and electroporated mixture placed onto mouse inactivated fetal fibroblasts in embryonic stem cell medium. After culture at 37° C., 5% CO₂ in air for 7 days, the cells formed distinct colonies with appearance similar to those of mouse embryonic stem cells. While we have not yet confirmed the presence of any stem cell markers in these cells, their morphology, characteristic colony growth and nuclear-to-cytoplasmic ratio are indicative of putative stem cells.

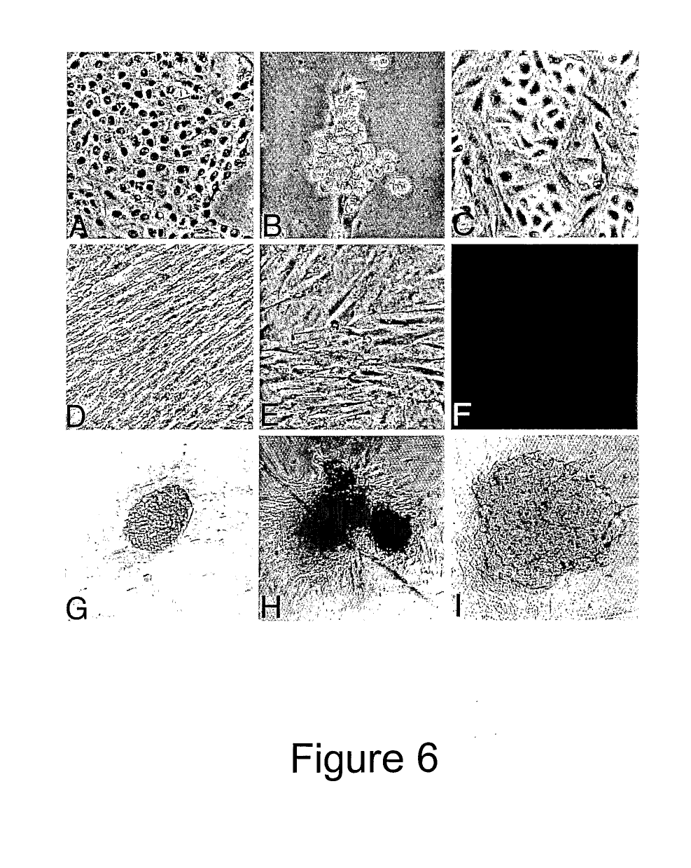

FIG. 6 contains the results of this experiment and shows bovine fetal pancreas primary cell culture 3 days after isolation. Cells either plated down (A) or remained in suspension in aggregates (B). Pancreatic cells four weeks after initiation of culture (C). Bovine fibroblast primary cell cultures (controls, D) were dissociated by trypsinization and electroporated with CytoTracker Blue (Molecular Probes, Eugene, Oreg.) prelabeled bovine oocyte lysate. After the electroporation, cells were plated on gelatin coated cell culture dishes and examined for the presence of CytoTracker Blue 24 hours later (E-phase, F-fluorescence using UV excitation). After 1 week in culture, the cells started forming colonies resembling stem cell aggregates (G), which increased in size during the following 2 weeks (H, I). All images were taken at 100×, recorded with DAGE-MTI camera and printed on a UVP printer. Images were scanned into Adobe Photoshop and pseudo-colored.

### REFERENCES

Blackmore C C et al., Emergency Radiol (1999) 212:117-125
Blank et al., Exp Cell Res 202, 224 (1992)
Blau and Baltimore, J Cell Biol 112, 781 (1991)
Blow J., J Cell Biol 122:993-1002 (1993)
Blow and Laskey, Cell 47, 577 (1986)
Bradley, Curr Opin Cell Biol 2, 1013 (1990)
Brill et al., Liver Biol Pathobiol 869 (1994)
Buschard and Rygaard, Acta Path Microbial Scand 84, 221 (1976)
Campbell et al., Rev Reprod 1, 40 (1996)
Diabetes Care 21:296-309 (1998)
Edlund, Curr Opin Cell Biol 11, 663 (1999)
Edlund, Diabetes 47, 1817 (1998)
Ernst R L et al., Am J Public Health (1994) 84:1261-1264
Freed C R et al., N Eng J Med (2001) 344:710-719
Grant et al., Lab Invest 67, 805 (1992)
Gutterman E M et al., JAGS (1999) 47:1065-1071
Hardeman et al., Cell 47, 123 (1986)
Harvey C et al., Paraplegia (1992) 30:834-850
Hoerger T J et al., Pharmacokinetics (1998) 14:541-557
Hinchcliffe et al., Science 283, 851 (1999)
See the Internet site of the U.S.C. D.C. regarding diabetes statistics.
Ivy M E and Cohen S M, Am J Emergency Med (1997) 15:591-597
Jacobson et al., Keystone Symposia, 313 (2001)
Kahan et al., Keystone Symposia, 117 (2001)
Kikyo and Wolffe, J Cell Sci 113, 11 (2000)
Kikyo et al, Keystone Symposia, 316 (2001)
Kikyo et al., Science 289, 2360 (2000)
Kirchhof et al., Biol Reprod 63, 1698 (2000)
Kopyov O V et al., Cell transplantation (1996) 5:327-337
Lake et al., Diabetes 38, 146 (1989)
Lanza and Chick, Ann NY Acad Sci 831, 323 (1997)
Lee et al., Nature Biotech 18, 675 (2000)
Leno et al., Cell 69, 151 (1992)
Levit K et al., Health Affairs (1998) 17:(6)99-110
Lumelsky N et al., see the Internet site of scienceexpress.org, Apr. 26, 2001
Lumelsky N et al., see the Internet site of scienceexpress.org, Apr. 26, 2001
Merriam, J Cell Sci 5, 333 (1969)
Morrison et al., Immunity 5, 207 (1996)
Odorico et al., Keystone Symposia, 323 (2001)
Pennisi and Vogel, Science 288, 1722 (2000)
Pesce and Scholer, Mol Reprod Dev 55, 452 (2000)
Povisen et al., Nature 248, 247 (1974)
Ricordi et al., Transplantation 45, 994 (1988)
Ricordi et al., Transplantation 52, 519 (1991)
Ricordi et al., In: Pancreatic islet cell transplantation 1, 132 (1994)
Roach et al., Eur Urol 23, 82 (1993)
Robertson, Teratocarcinomas and embryonic stem cells: a practical approach (1987)
Rodriguez-Boulan and Zurzolo, J Cell Sci 17, 9 (1993)
Sander and German, J Mol Med 75, 327 (1997)
Schöler et al., Cell 66, 291 (1991)
Schuldiner et al., Keystone Symposia, 130 (2001)
Schuldiner et al., Keystone Symposia, 203 (2001)
Schuldiner et al., Proc Natl Acad Sci USA 97, 11307 (2000)
Soria et al., Diabetes 49, 157 (2000)
St-Onge et al., Curr Opin Genet Dev 9, 295 (1999)
Theise et al., Hepatology 32, 11 (2000)
Thomson et al., Proc Natl Acad Sci USA 92, 7844 (1995)
Thomson et al., Science 282, 5391 (1998)
Thomson and Marshall, Curr Top Dev Biol 38, 133 (1998)
Tuch et al., Diabetes 33, 1180 (1984)
Vogel, G, Science (2001) 292:415
Xu et al., Keystone Symposia, 133 (2001)

What is claimed:

1. A method for effecting de-differentiation of a somatic cell comprising
(a) culturing a somatic cell in the absence of growth factors, cytokines, or other differentiation-inducing agents,
(b) introducing components of cytoplasm of plutipotent cells into the somatic cell; and
(c) allowing the cell to de-differentiate.

2. The method of claim 1, wherein the cell is a mammalian somatic cell selected from the group consisting of fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, macrophages, monocytes, and mononuclear cells.

3. The method of claim 1, wherein step (a) comprises culturing the cell in serum-free medium.

4. The method of claim 1, wherein the pluripotent cells are selected from the group consisting of oocytes, blastomeres, inner cell mass cells, embryonic stem cells, embryonic germ cells, embryos consisting of one or more cells, embryoid body (embryoid) cells, morula-derived cells, teratoma (teratocarcinoma) cells, as well as multipotent partially differentiated embryonic stem cells taken from later in the embryonic development process.

5. The method of claim 1, wherein the pluripotent cells are oocytes.

6. The method of claim 5, wherein the oocytes are metaphase II oocytes.

7. The method of claim 5, wherein the oocytes are Xenopus oocytes.

8. The method of claim 1, further comprising centrifuging oocyte cytoplasm, and isolating a fraction of the centrifuged oocyte cytoplasm containing the components of cytoplasm of step (b).

9. The method of claim 1, wherein step (b) comprises placing the somatic cell in solution containing components of cytoplasm of pluripotent cells, and introducing components of cytoplasm of plutipotent cells into the somatic cell by electroporation.

10. The method of claim 1, further comprising, after the step of introducing components of cytoplasm of plutipotent cells,
culturing the cell under conditions suitable for maintaining pluripotent stem cells in an undifferentiated state

11. The method of claim 1, further comprising, after the step of introducing components of cytoplasm of plutipotent cells,
culturing the cell under conditions that induce or direct partial or complete differentiation to a particular cell type

12. The method of claim 12, comprising, after the step of introducing components of cytoplasm of plutipotent cells,
culturing the cell in medium containing nerve growth factor.

13. The method of claim 12, comprising, after the step of introducing components of cytoplasm of plutipotent cells,
culturing the cell in DMEM/F12 ITS medium that contains nerve growth factor.

14. A method for reprogramming a somatic cell to become a cell of neural lineage, comprising:
(a) culturing a somatic cell that is not of neural lineage in the absence of growth factors, cytokines, or other differentiation-inducing agents,
(b) introducing cytoplasm of a pluripotent cell into the cell; and
(c) culturing the cell in medium containing nerve growth factor.

15. The method of claim 14, wherein step (c) comprises culturing the cell in DMEM/F12 ITS medium that contains nerve growth factor.

16. The method of claim 14, further comprising assaying to detect a marker of cells of neural lineage.

17. A composition of cells of neural lineage prepared by the method of claim 14.
